(19) United States
(12) Patent Application Publication
(10) Pub. No.: US 2014/0180209 A1
(43) Pub. Date: Jun. 26, 2014

(54) Syringe needle catching device and safety syringe with this device

(71) Applicant: Jan Kavalirek, Praha 7 (CZ)

(72) Inventor: Jan Kavalirek, Praha 7 (CZ)

(21) Appl. No.: 14/123,205

(22) PCT Filed: Jun. 7, 2012
(86) PCT No.: PCT/IB2012/001583

§ 371 Date: Mar. 4, 2014

(30) Foreign Application Priority Data
Jun. 3, 2011 (CZ) PV 2011-335

(51) Int. Cl. A61M 5/32

(52) U.S. Cl. CPC A61M 5/3221
USPC 604/110

(57) ABSTRACT

A catching device of a needle for a safety syringe includes a tube containing an inserted connection foot of the needle or an inserted hub adapted for fitting of the connection foot of the needle where in the connection foot of the needle or in a hub a cylindrical cavity with a conical inclination is created and the tube. An inserted plunger fitted on the front face with a conical adapter with a forward-running cylindrical projection and where at the same time in the foot of the needle or in the hub adapted for fitting of the connection foot of the needle a transversal slot is created. The inserted plate is designed as a catching plate that has a circular opening in its centre where the perimeter edge of this circular opening is adapted to increased elasticity in the radial direction by creating of an inner blade (3410) in the form of a conical skirt, getting narrower in the direction outwards from the syringe tube.

### FIELD OF THE INVENTION

The invention deals with syringes, in particular with catching devices where after use the needle is retracted into the syringe cavity and is also protected from subsequent extension while the retraction is enabled by a device for connection of the needle with the plunger with the use of which the needle is then retracted into the syringe cavity. In particular it deals with a device that ensures locking of the needle in the functional position during aspiration of the active substance into the syringe and subsequently during expelling of the active substance when it is applied where after that the needle must be caught by the plunger and retracted into the syringe barrel or tube for the purpose of protected storage of the needle until safe disposal of the syringe as a whole as a syringe with a needle, individually or as a whole represents hazardous waste.

### BACKGROUND OF THE INVENTION

At present, syringes are known where the needle is fixed in the syringe tube while after emptying of the syringe content, when the plunger is moved towards the needle, the back part of the needle is caught by the front face of the plunger using the principle of a reverse hook or hooks while the return movement of the plunger retracts the needle into the syringe cavity while at the same time the fixation of the needle or needle carrier in the syringe tube is being released or overcome so the syringe can be subsequently pulled by the plunger into the syringe cavity, where it is then safely protected from contact with external object. This is also the main purpose of the design of the described type while the protection is important with regard to protection of persons from injury by otherwise a freely protruding needle tip and mainly from possible infection that may be caused by such injury.

Known designs use various approaches to fixation of the needle in the syringe shell and attachment of the needle to the plunger face, which should retract the needle into the syringe cavity during its return movement. E.g. in the file U.S. Pat. No. 5,405,327 the needle is mounted in the tube with the use of an inserted O-ring, which is simple, but may not always protect the needle from undesired pushing into the syringe during sticking of the needle where such undesired pushing inside results in spoilage of the whole filled syringe. According to the file CZ PV 1996-3167 the needle is fixed in the syringe in a similar way with the only difference that the support ring is directly created on the inner surface of the socket for fitting of the needle, so analogous risk of premature retraction of the needle into the syringe can be expected in this case as well. The needle is similarly anchored in the syringe tube, e.g. in the design according to the file U.S. Pat. No. 5,417,661. A more reliable attachment of the needle in the syringe tube is achieved in the syringe design in accordance with the file CZ PV 1996-3021, where the needle is fixed in the tube with the use of a lock, referred to as a split pin in the above mentioned file where this pin is deformed during catching of the needle by a projection on the plunger face, being disengaged from a groove on the inner side of the tube, which causes release of the needle in the tube so that it can be subsequently easily retracted into the syringe cavity. As regards catching of the needle by the plunger, such designs are known when a projection is created on the plunger, namely a projection with edges based on the principle of reverse hooks cooperating with an edge of the needle. The latter designs are known among other things from the files CZ 1996-3021, CZ 1996-3167, U.S. Pat. No. 5,405,327. Here, in the last mentioned files a mushroom-shaped projection is used, which is pushed with its head for the connection with the needle into a cavity in the needle base or in the carrier of this needle base respectively. Additionally, in the design of CZ 1996-3021 the mushroom shape is modified into the shape of a cone fitted with a cylindrical projection that is introduced into the corresponding cavity created in the needle base or in this case on the carrier of this base. Here, however, the problem of insertion of this cylindrical part into the above mentioned cavity must be solved. This insertion, in spite of subsequent fitting with a certain play, may cause problems, as firstly generally quite precise coaxial alignment of the above mentioned cylindrical part with regard to the corresponding cavity in the needle base or in the carrier of this base cannot be guaranteed and secondly to achieve intentional misalignment of the needle stored subsequently in the syringe cavity the projection and consequently the cylindrical part running out of it towards the front is often intentionally shaped with coaxial misalignment in relation to the above mentioned cavity. An existing design, namely that one described in the file CZ PV 1996-3021 proposes that the end of the cylindrical part projecting forwards should be symmetrically rounded. It is true that this will help, moreover in cooperation with a conical inclination leading to the corresponding cavity, to guide the cylindrical end of the projection into the corresponding cavity, but still in some cases some blocking at the inlet of this cavity or breaking of the front part of the cylindrical projection may occur. This inconvenient situation will then be the more probable, the larger the angle of needle deflection after retraction into the syringe cavity is selected, or the larger the corresponding angle of deflection of the projection with the cylindrical forward-running part is selected. But for higher protection against repeated extension of the needle experience shows that in a design of this type the hitherto most frequently used angles of approx. 15 degrees of deflection from the longitudinal axis of the syringe are not quite sufficient. Therefore, larger deflection angles tend to be selected, which however increases the risk of breaking or blocking of the end of the above mentioned cylindrical, forward-running part. This shortcoming is resolved with a modification of the end of the cylindrical, forward-running part, namely a modification described in CZ U 17541, where this cylindrical projection features an oblique, preferentially plane chamfer at its free end. This allows you to select a larger angle of deflection of the cylindrical projection from the longitudinal axis of the syringe without increasing the risk of blocking or breaking of this projection. In the last mentioned design, which also contains a lock plate mounted transversally in the slot of the needle hub or needle foot as the locking element of the needle or needle hub or carrier, in the last stage of insertion of the plunger into the syringe tube, i.e. at the end of application of the active substance with the syringe, increased resistance is produced caused by deflection of the above mentioned plate as well as finally by impression of the conical adapter of the plunger into the bottom narrowed opening in the needle foot or needle hub. This results in the danger that the user will not completely push the plunger to the position where this resistance is overcome and when the edge of the above mentioned conical adapter only snaps behind the narrowed bottom margin of the cavity in the needle foot or needle hub. Then, the needle cannot be retracted into the syringe tube and if the user does not make further attempts to catch and retract the needle, the syringe will remain in the waste with the needle unprotected, which is very dangerous. To facilitate interconnection of the conical adapter of the plunger with the needle foot or hub or needle carrier in general it is then desirable to take a measure that would facilitate insertion of the above mentioned parts into each other on the one hand, but that would not compromise the strength of the connection produced this way so that reliable catching and retraction of the needle can still be guaranteed on the other hand. If such a measure meant shape modifications of the bottom part of the plunger adapter, a modification of a similar syringe is mentioned in U.S. Pat. No. 4,888,002, where on a conical adapter of the plunger peripheral projections are created having however the character of a kind of thread where the connection is established by screwing. Here, the needle hub is also modified in such a way that it contains annular grooves along the outer perimeter the task of which is sealing and fixation of this part in the syringe tube in this case and at the same time corresponding annular grooves can be seen on the outer part of this hub, which are however used for mutual fixed interconnection during the production of the hub and cannot thus be considered as a solution of the above mentioned set task of the presented invention consisting in modification of the plunger adapter to improve the interaction in relation to the needle hub or needle foot. An improvement of the above mentioned plunger adapter to ensure better interaction with the needle hub is presented in the file CZ 302349, where the margin of the conical adapter is designed in such a way to ensure higher elasticity and the locking plate is adapted along its free edges by chamfering or rounding to improve the movement of this plate in the groove, especially in a position with a higher deflection of this locking plate during the insertion of the conical adapted of the plunger through the opening in this plate. This solution improves the force proportion for easier insertion of the conical adapter of the plunger into the cavity in the needle hub and for firmer anchoring of this adapter in the hub, but from the technological point of view there is a disadvantage that the design of the plunger adapter and at the same time the production process are becoming excessively complex. However, the previous solution of the same problem, where the plunger adapter is rather simpler, both in the conical or semicircular version, exhibits a disadvantage consisting in a more complicated production of the mould consisting in a more complex sequence of assembly and disassembly of the mould during the entire plastic-moulding production as in the needle hub a partly closed cavity needs to be produced that is delimited at the inlet by the very edge where the plunger adapter gets caught against return movement and extension.

Thus, the task of the presented invention is to create such a system of the needle plunger and hub where the inconvenient partly closed cavity will not have to be employed.

### SUMMARY OF THE INVENTION

The above mentioned disadvantages and the described purpose is achieved in line with the presented invention with a catching device of a needle for a safety syringe containing a tube and in the tube containing an inserted connection foot of the needle or inserted hub adapted for fitting of the connection foot of the needle where in the connection foot of the needle or in the hub a cylindrical cavity with a conical inclination is created and the tube further contains an inserted plunger fitted on the front face with a conical adapter with a forward-running cylindrical projection and where at the same time in the needle foot or in the hub adapted for fitting of the connection foot of the needle a transversal slot is created in which a plate is inserted and where the principle of the catching device is that the inserted plate is designed as a catching plate that has a circular opening in its centre where the perimeter edge of this circular opening is treated in such a way to exhibit increased elasticity in the radial direction by creation of an inner blade in the form of a conical skirt getting narrower outwards from the syringe tube. This arrangement reduces the resistance during penetration of the perimeter edge of the conical adapter into the above mentioned circular opening or the conical inclination in the needle foot or hub without compromising the strength of this perimeter edge or inner blade in any way, which is important for transmission of forces during the retraction of the needle into the cavity in the syringe tube. It is also convenient if the conical adapter of the plunger is attached to the plunger with the use of a cylindrical neck with a diameter corresponding to 40% to 60% of the diameter of the base of this conical adapter of the plunger where the ratio of the height of this neck to the diameter of this neck is from 2.5 to 5.0. This design facilitates deflection of the needle, carried by the conical adapter of the plunger, namely after retraction into the cavity in the syringe tube. What is also convenient for the conical adapter is if this conical adapter of the plunger is axially misaligned at the fixation point to the plunger via the cylindrical neck in relation to the longitudinal axis of the cylindrical cavity in the needle foot or to the longitudinal axis of the cylindrical cavity in the hub. Thus, in an otherwise well-known way the above mentioned deflection of the needle after retraction into the cavity in the syringe tube is achieved while the amount of axial misalignment of the conical adapter of the plunger is 10 to 20 degrees in a convenient embodiment. As compared to some well-known designs a rather smaller axial misalignment is selected, which has proved to be sufficient for the deflection of the needle and at the same time a certain reduction of forces has been achieved during the overcoming of this axial misalignment during the insertion of this conical adapter into the corresponding cavity in the needle foot or in the hub. This leads, in combination with reduction of these forces through other measures in accordance with this invention, to generally easier introduction of the conical adapter of the plunger into the corresponding cavity as mentioned above. The cylindrical projection of the conical adapter then conveniently features an oblique chamfer at its free end, designed as a plane chamfer, namely created at the angle of 30 to 60 degrees in relation to the plane that is perpendicular to the longitudinal axis of this cylindrical projection. This modification, reducing the value of the amount of chamfering as compared to some existing similar designs, also achieves further reduction of the risk of jamming or risk of increased resistance during the insertion of the cylindrical projection into the corresponding opening in the needle foot or hub. Conveniently, the needle hub is then mounted in the socket of the syringe tube with the use of an outer perimeter projection fitting in an inner perimeter groove in this socket, where the outer axial contact surfaces between the hub and the socket, closer to the socket orifice are created on the plane perpendicular to the longitudinal axis of the syringe while the inner axial contact surfaces between the hub and socket, further away from the socket orifice are created in the shape of conical surfaces with the apex of such a cone closer to the syringe plunger. The invention further deals with a safety syringe containing the above mentioned catching device where the principle of the syringe is that on the inner side of the syringe tube an inner perimeter projection is created before the front edge of the plunger corresponding to such a position of the plunger in the syringe tube cavity where the perimeter edge of the base of the conical adapter of the plunger is found before the catching plate and at the same time the front end of the cylindrical projection of this conical adapter is found behind the catching plate. This way a safety syringe is created with the above mentioned catching device where the inner perimeter projection on the inner side of the syringe tube additionally prevents undesired premature insertion of the conical adapter of the plunger behind the catching plate, especially in a situation when the fitted needle of this syringe is pushed into an ampoule with the substance that should be aspirated and subsequently injected and where impression of the needle by the plunger pressure cannot be excluded. The above mentioned inner perimeter projection in the syringe tube has such a size and shape enabling after overcoming of increased resistance movement of the plunger to the position where the conical adapter is pushed behind the catching plate, which happens just after the completion of discharge of the substance from the syringe, which is followed by the final retraction of the needle into the cavity in the syringe tube.

So this way one goal is achieved through a combination of more adaptations, namely while maintaining the required reliability of blocking the needle foot or hub in the syringe during its active function to adequately facilitate insertion of the conical adapter of the plunger into the cavity in the foot of the needle or hub and to reliably catch and connect this needle foot or this hub to the plunger to subsequently retract the needle into the syringe tube cavity and all this with a simplified design as well as the working cycle of the mould for production of mainly the needle hub. We must point out that in this design the purpose of the catching plate is not to fix the needle hub in the syringe tube socket as it is in the case of the locking plate in similar designs described in files CZ 301122 and CZ 302349, but the function of this catching plate in the embodiment based on the invention consists in catching of the needle by the conical projection of the plunger while unlike other designs within the hitherto state of the art where the catching blades and grooves are created directly in the cavity of the entire body of the hub or around the perimeter of the conical projection of the plunger in the embodiment based on the invention a blade is created along the perimeter of the circular opening of the catching plate inserted into the basic body of the needle hub. This is why during the entire moulding of the above mentioned catching plate a relatively more complex shape of the inner blade along the circular opening in this catching plate can be created relatively easily from the technological point of view. It is true that the necessity to produce such a catching plate as a separate part and to additionally install it into the main body of the needle hub represents a certain disadvantage, but at a certain extent of serial production the advantages of simpler production of the mould and easier moulding technology when such a mould is used will finally prevail. Finally, another benefit is if from the orifice of the socket cavity towards the inner perimeter groove for mounting of the hub in this socket along a part of this section in this cavity an inner thread is created, corresponding to the outer thread created on the foot of the cooperating needle, namely in the form for connection of the Luer Lock type. When this type of needle fitting, i.e. a screwing movement, is used, during the fitting of the needle perimeter forces are generally applied, which reduces the risk of inadvertent impression of the needle hub into the syringe cavity due to mostly axial forces present in the stage of syringe preparation.

### BRIEF DESCRIPTION OF THE DRAWINGS

The presented invention is further described in a more detailed way and clarified with the use of a sample embodiment, also with the support of attached drawings where

FIG. 1 shows a detail of the conical adapter with the cylindrical projection in the position of insertion into the corresponding hub cavity, namely in an axial cross-section,

FIG. 2a presents the entire body of the needle hub in a perspective view, FIG. 2b shows the same hub in the ground plan view, FIG. 2c shows the same hub again illustrated in a transversal vertical cross-section and FIG. 2d shows the same hub, this time in a longitudinal vertical cross-section while further

FIG. 3a illustrates the entire catching plate of the conical adapter of the plunger, namely in a perspective view, FIG. 3b shows the same catching plate in the ground plan projection, FIG. 3c shows the same catching plate again illustrated in a transversal vertical cross-section and FIG. 3d shows the same catching plate, this time in a longitudinal vertical cross-section while

FIG. 4 presents an expanded perspective view of the syringe with the plunger,

FIG. 5 illustrates the complete syringe unit before application, in particular in the plunger position during aspiration of the substance for application in an axial cross-section and

FIG. 6 shows, in an axial cross-section again, the whole syringe unit after application and after catching of the needle hub by the conical projection and finally

FIG. 7a presents the syringe in the situation after retraction of the hub with the needle into the syringe cavity in an axial cross-section again.

### DETAILED DESCRIPTION OF THE INVENTION

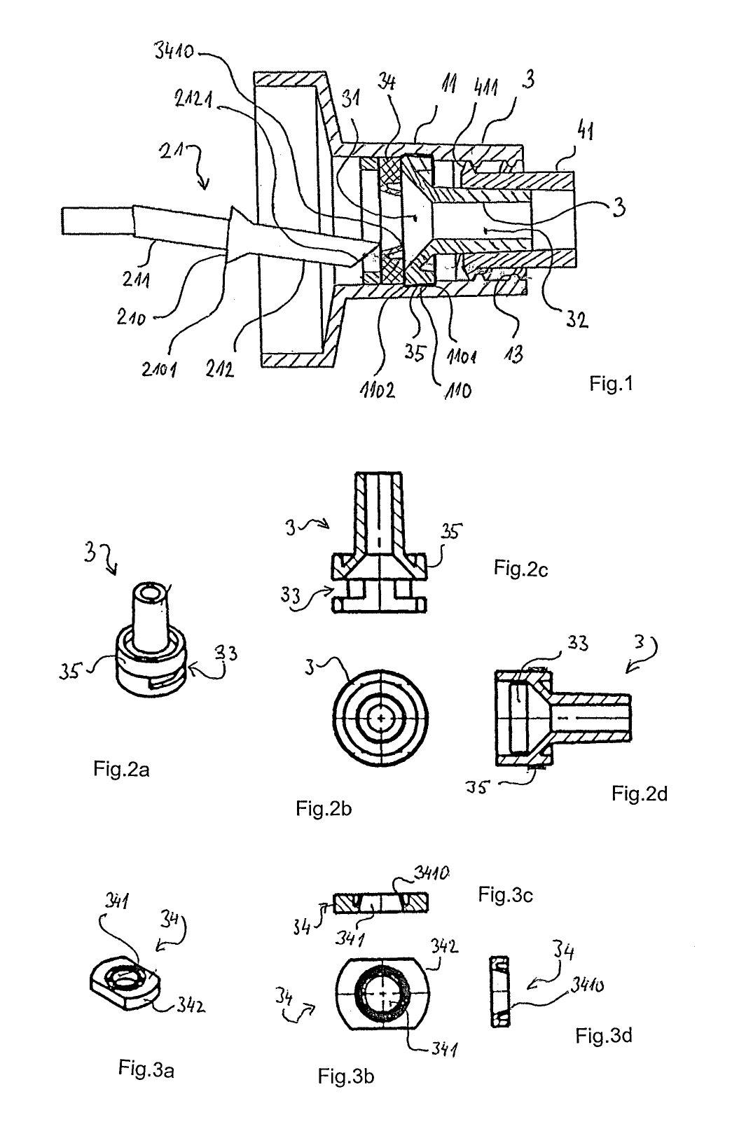

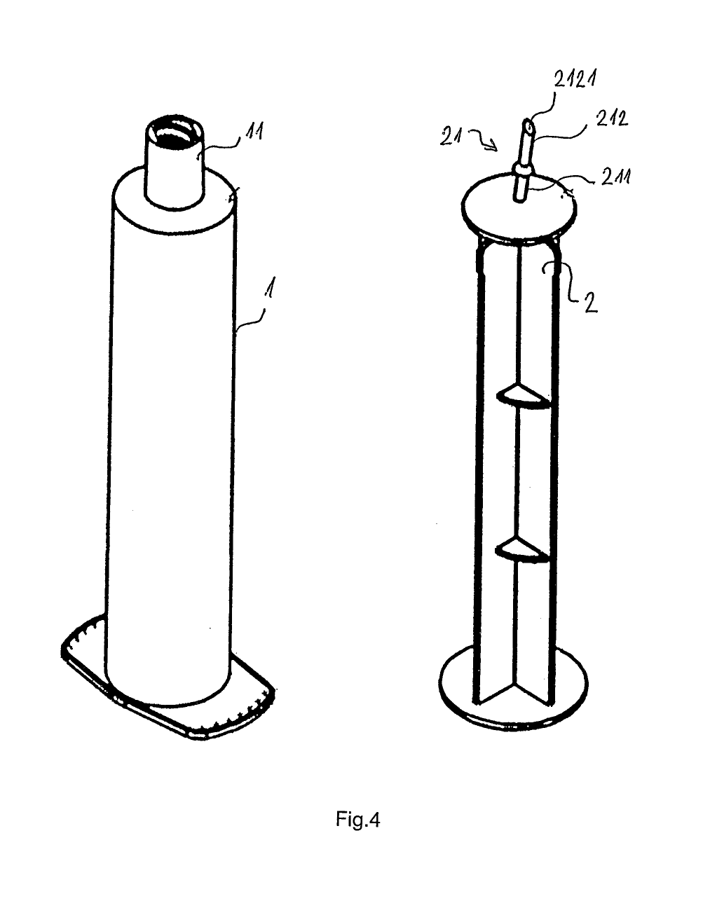

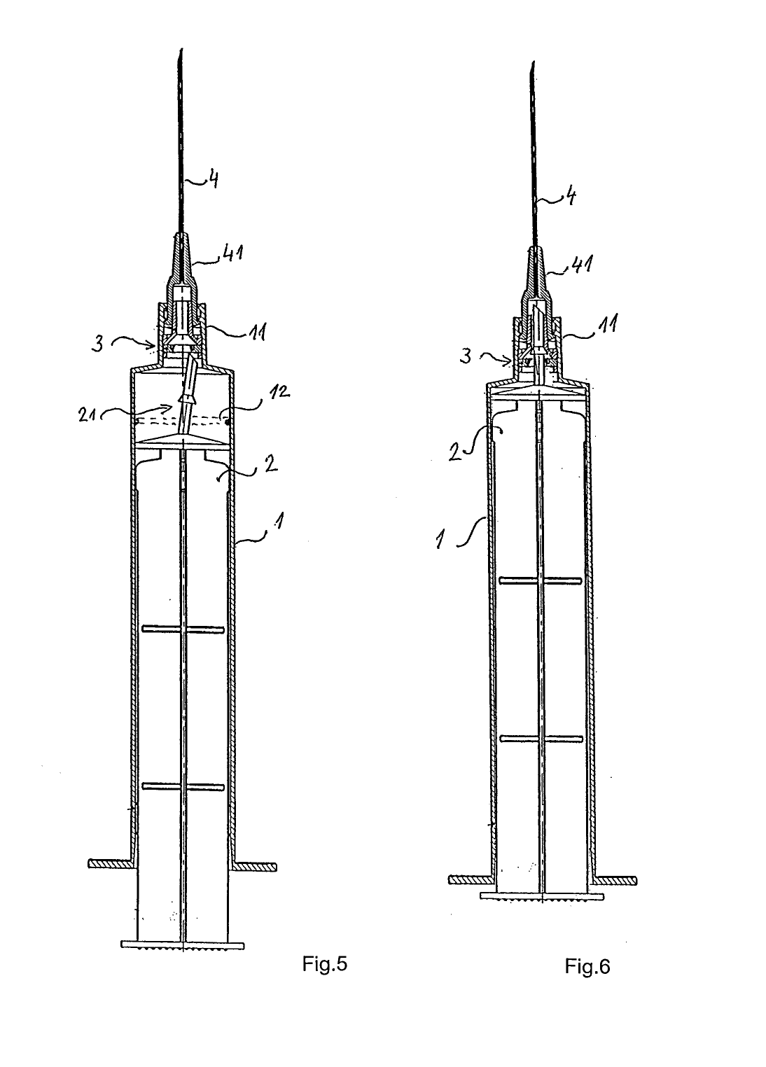

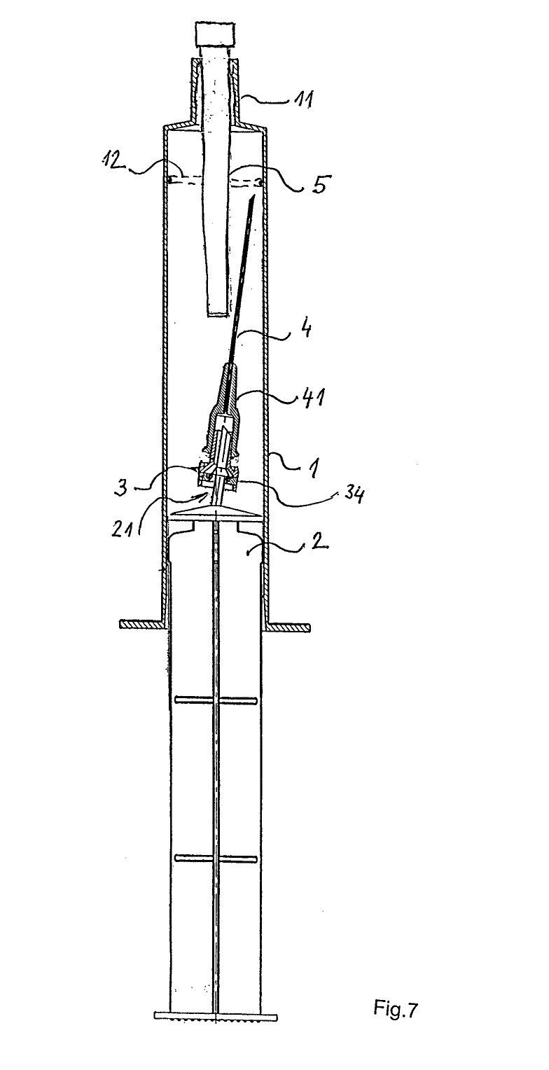

In the sample embodiment the syringe consists of a tube 1 in which a plunger 2 is fitted and in the front part of the syringe tube 1, here in a narrowed socket 11 the hub 3 of the foot 41 of the needle 4 is inserted. In this hub 3, symmetrically with its longitudinal axis, a cylindrical cavity 32 with a conical inclination 31 is created. On the front face of the plunger 2 a conical adapter 21 is created that is connected to the plunger 2 via a neck 211. From the front part of the conical adapter 21 a cylindrical projection 212 runs. This cylindrical projection 212 is chamfered at its free end with a plane chamfer 2121, where the plane of this chamfer 2121 makes the angle of 60 degrees with the plane perpendicular to the longitudinal axis of the corresponding cylindrical projection 212. Further, the cylindrical projection 212 of the conical adapter 21 of the plunger 2 is designed in such a way that the size of the cylindrical projection 212 corresponds to 10% of the diameter of the base 210 of the conical adapter 21. The conical adapter 21 of the plunger 2 is attached to this plunger 2 via a cylindrically shaped neck 211 with a diameter corresponding to 25% of the diameter of the base 210 of the conical adapter 21 of the plunger 2 while at the same time the ratio of the height of this neck 211 to the diameter of this neck 211 is 4.0. As regards the conical adapter 21 of the plunger 2, the conical adapter 21 of the plunger 2 is also axially misaligned in its fixation to the plunger 2 via the neck 211, considered in relation to the longitudinal axis of the cylindrical cavity 32 in the hub 3. At the same time the axial misalignment of this conical adapter 21 of the plunger 2 in the unloaded condition amounts to 15 degrees in this sample embodiment. From the point of view of the general design of fitting of the needle 4 in the syringe tube 1, namely in the illustrated sample embodiment with fitting of the foot 41 of the needle 4 in the syringe tube 1 via the hub 3 inserted in the syringe tube 1 the figures also show that in the hub 3 adapted for fitting of the connection foot 41 of the needle 4 a transversal slot 33 is created in which a catching plate 34 is inserted that has a circular opening 341 in its centre and with its free ends 342 it reaches to the walls of the cavity in the socket 11 of the syringe tube 1. The syringe, or needle 4, respectively, is also equipped with a cover or bushing 5 that protects the needle 4 and/or the entrance into the cavity of the syringe tube 1 from damage and contamination in the stage before the use of this syringe. After the use of the syringe this bushing 5 can be conveniently inserted with its narrower end into the socket 11 of the syringe tube 1, which will close the space in the syringe tube 1 on the one hand and on the other hand the needle 4 is additionally protected from later possible extension, which is always undesirable in the subsequent stages. Finally, we would like to refer to the detail of the front part of the syringe with the hub 3 in the socket 11 and with the fitted needle 4 in FIG. 1, which shows the established connection of the foot 41 of the needle 4 with the hub 3 of the needle 4 with an outer thread 411 on the foot 41 of the needle 4 and with an inner thread 13 in the cavity of the socket 11 where this connection is usually referred to as a Luer Lock. As regards the whole safety syringe the sample embodiment also illustrates a convenient arrangement inside the syringe tube 1 where on the inner side of this syringe tube 1 an inner perimeter projection 12 is created before the front edge of the plunger 2, namely in the place corresponding to such a position of the plunger 2 in the cavity of the syringe tube 1 where the perimeter edge 2101 of the base 210 of the conical adapter 21 of the plunger 2 is found before the catching plate 34 and at the same time the front end of the cylindrical projection 212 of this conical adapter 21 is found behind the catching plate 34. The inner perimeter projection 12 created on the inner side of the syringe tube 1 in this position additionally prevents inadvertent premature insertion of the conical adapter 21 of the plunger 2 behind the catching plate 34.

The function of the complete device created in accordance with the described sample embodiment is as follows. The needle 4 is fitted onto the socket 3 and if the syringe is not supplied as filled, then the liquid for application is aspirated from an ampoule or a liquid sample is drawn by aspiration while after that the aspirated liquid is applied or possibly the drawn sample is discharged into a transport ampoule. At the end of the discharge the cylindrical projection 212 is first inserted into the conical inclination 31 and then into the cylindrical cavity 32 in the hub 3 where finally the perimeter edge 2101 of the base 210 of the conical adapter 21 of the plunger 2 snaps behind the inner blade 3410 created along the perimeter of the circular opening 341 in the catching plate 34. So during the penetration of the conical adapter 21 through the conical inclination 31 the conical adapter 21 first passes through the circular opening 341 in the catching plate 34 while after that the perimeter edge 2101 of the conical adapter 21 deforms the inner blade 3410 of the circular opening 341 of the catching plate 34 and subsequently, during the return movement the plunger 2 retracts the hub 3 carrying the fitted needle 4 with it into the syringe tube 1. The hub 3 itself is fixed in the cavity of the socket 11 with the use of its outer perimeter projection 35 fitting into the inner perimeter groove 110 in the socket 11 while undesired disengagement of the hub 3 from the socket 11 is prevented by the arrangement where the outer axial contact surfaces 1101 between the hub 3 and socket 11 closer to the orifice of the socket 11 are created on the plane that is perpendicular to the longitudinal axis of the syringe while the inner axial contact surfaces 1102 between the hub 3 and the socket 11, further away from the orifice of the socket 11 are designed with the shape of conical surfaces with the apex of such a cone closer to the syringe plunger. Through the selection of the overlap of the outer perimeter projection 35 and at the same time by selecting the angle at the inner axial contact surface 1102 you can achieve an optimum force proportion between the requirement for relatively firm mounting of the hub 3 in the socket 11 on the one hand and on the other hand the requirement for still acceptable force necessary to release this mounting at the beginning of retraction of the hub 3 with the needle 4 into the cavity in the syringe tube 1. After retraction of the needle 4 into the cavity of the tube 1 the needle 4 is deflected from the straight axial direction thanks to axial misalignment of the adapter 21 of the plunger 2. In such a deflection the needle 4 cannot be extended again, which is the main purpose of this type of syringes. Finally, the opening in the socket 11 is blinded with the reversely applied or inserted bushing 5. As mentioned above, premature insertion of the conical adapter 21 into the catching plate 34 at the beginning of the entire process is prevented by the inner perimeter projection 12 in the tube 1, which the front edge of the plunger 2 leans against during the impression of the syringe needle 4 into the ampoule. To achieve the final goal, i.e. protection of the needle, at the beginning of the whole process the needle 4 or the needle 4 with the hub 3 must be protected from being pushed into the cavity in the syringe tube 1, which is ensured here by fitting of the hub 3 in the inner perimeter groove 110 of the socket 11 and also by the above mentioned arrangement consisting in creation of the inner perimeter projection 12 in the syringe tube 1 as well as reliable release of the hub 3 from the syringe tube 1 before retraction into its cavity, which is achieved here by unlocking of this hub 3 during the return pull, when the outer perimeter projection 35 of the hub 3 is disengaged from the inner perimeter groove 110 in the socket 11 and for reliable guiding during insertion of the cylindrical projection 212 into the conical inclination 31 of the cylindrical cavity 32 of the hub 3, or mainly to the entire cylindrical cavity 32, respectively, it is important for this cylindrical projection 212 to mainly have such a shape of its end that will facilitate, even though there may be certain axial misalignment, this retraction and at the same time it will be as resistant as possible to jamming at the entrance of the above mentioned cylindrical cavity 32 on the one hand and as resistant as possible to breaking of the end of the cylindrical projection 212 on the other hand. Optimization of the shape of the end of the cylindrical projection 212 or the resulting optimum limits of such a shape are well-known as such while the parameters of this design are further optimized in this case. Besides the basic idea of facilitated connection of the needle 4 to the plunger 2 using the arrangement with a separately produced catching plate 34 what is simplified here is the production technology and operation of the mould for the hub 3 consisting here of the main body and the catching plate 34 inserted in the assembled condition into the transversal slot 33 of the hub 3. This arrangement then conveniently, as a whole, works with the facilitated catching of the needle 4 in combination with other features that are the object of other improvements within the presented invention. However, what is the most important is the contribution in the sphere of safe disposal of used syringes where the facilitated catching of the needle 4 by the catching device to retract it into the cavity of the syringe tube 1 eliminates such cases when after the first failed attempt at such catching and retraction of the needle 4 the syringe is finally disposed of with the needle 4 extended. This facilitated retraction of the needle 4 is a consequence of the basic technical features of the presented invention, is supported and improved in modifications containing other convenient technical features and is accompanied by a substantial advantage in the sphere of the production technology as well as operation of the injection mould for the hub 3 of the needle 4.

### INDUSTRIAL APPLICABILITY

The device can be used as a safe single-use syringe while it can be applied everywhere a cheap, but reliable design preventing injury by a syringe needle is required, including protection from repeated extension of the protected needle. What is important is its contribution to safety within the disposal of used syringes where the facilitated catching of the needle by the catching device ensuring retracting of the needle into the syringe tube cavity such cases are eliminated when after the first failed attempt at such catching and retraction of the needle the syringe is finally disposed of with the needle extended. More convenient production technology and operation of the mould enabled by the improved design of the hub results in a reduction of production costs.

1. A catching device of the needle (4) for a safety syringe comprising:
a tube (1) including an inserted connection foot (41) of the needle (4) or an inserted hub (3) adapted for fitting of the connection foot (41) of the needle (4) where in the connection foot (41) of the needle (4) or in the hub (3) a cylindrical cavity (32) with a conical inclination (31) is created and the tube (1) further contains an inserted plunger (2) fitted on the front face with a conical adapter (21) with a forward-running cylindrical projection (212), and where at the same time in the foot (41) of the needle (4) or in the hub (3) adapted for fitting of the connection foot (41) of the needle (4) a transversal slot is created (33) in which a plate is inserted the plate is further defined as a catching plate (34) that has a circular opening (341) in its centre where the perimeter edge of this circular opening (341) is adapted to increased elasticity in the radial direction by creating of an inner blade (3410) in the form of a conical skirt, getting narrower in the direction outwards from the syringe tube (1).

2. The catching device in accordance with claim 1, wherein the conical adapter (21) of the plunger (2) is attached to the plunger (2) via a cylindrical neck (211) with a diameter corresponding to 40% to 60% of the diameter of the base (210) of this conical adapter (21) of the plunger (2), where the ratio of the height of this neck (211) to the diameter of this neck (211) is 2.5 to 5.0.

3. The catching device in accordance with claim 1 wherein the conical adapter (21) of the plunger (2) is axially misaligned at the place of fixation to the plunger (2) via the cylindrical neck (211) in relation to the longitudinal axis of the cylindrical cavity in the foot (41) of the needle (4) or to the longitudinal axis of the cylindrical cavity (32) in the hub (3) while the amount of axial misalignment of the conical adapter (21) of the plunger (2) is 10 to 20 degrees.

4. The catching device in accordance with any of claims 1 wherein the cylindrical projection (212) of the conical adapter (21) has an oblique chamfer (2121) at its free end designed as a plane chamfer and created at the angle of 30 to 60 degrees in relation to the plane perpendicular to the longitudinal axis of this cylindrical projection (212).

5. The catching device in accordance with any of claims 1 wherein the hub (3) of the needle (4) is fitted in the socket (11) of the syringe tube (1) with the use of an outer perimeter projection (35) fitting into the inner perimeter groove (110) in this socket, where the outer axial contact surfaces (1101) between the hub (3) and the socket (11), closer to the orifice of the socket (11), are created on the plane perpendicular to the longitudinal axis of the syringe while the inner axial contact surfaces (1102) between the hub (3) and the socket (11), further away from the orifice of the socket (11), are created in the shape of conical surfaces with the apex of such a cone closer to the plunger (2) of the syringe.

6. The catching device in accordance with any of claims 1 wherein from the orifice of the cavity of the socket (11) towards the inner perimeter groove (110) for mounting of the hub (3) in this socket (11) along a part of this section an inner thread (13) is created in this cavity corresponding to the outer thread (411) created on the foot (41) of the cooperating needle (4), having the shape, course and termination of the threads (13,411) adapted for a connection of the Luer Lock type.

7. A safety syringe containing a needle catching device wherein on an inner side of a syringe tube (1) an inner perimeter projection (12) is created before a front edge of the plunger (2) while this place corresponds to such a position of the plunger (2) in the cavity of the syringe tube (1) where a perimeter edge (2101) of a base (210) of a conical adapter (21) of the plunger (2) is found before a catching plate (34) and at the same time the front end of a cylindrical projection (212) of this conical adapter (21) is found behind the catching plate (34).
